(19) United States
(12) Patent Application Publication
(10) Pub. No.: US 2011/0172448 A1
(43) Pub. Date: Jul. 14, 2011

(54) PROCESS FOR CARRYING OUT HETEROGENEOUSLY CATALYZED REACTIONS WITH HIGH SELECTIVITY AND YIELD

(76) Inventors: Martin Votsmeier, Leuztelsachsen (DE); Susanne Ungermann, Linsengericht (DE); Juergen Gieshoff, Gelnhausen (DE); Thomas Kreuzer, Karben (DE)

(21) Appl. No.: 13/000,822

(22) PCT Filed: Jun. 24, 2009
(86) PCT No.: PCT/EP2009/004557

§ 371 Date: Mar. 16, 2011

(30) Foreign Application Priority Data
Jun. 27, 2008 (EP) 08159300.6

(51) Int. Cl. C07D 303/04; C07C 45/27; C07C 45/34; C07C 45/00; C07C 51/00; C07C 17/00; C07C 29/00; C07C 15/02

(52) U.S. Cl. 549/513; 568/383; 568/401; 568/449; 562/598; 570/123; 552/208; 562/493; 568/835; 562/607; 570/216; 585/400

(57) ABSTRACT

A cost effective process is presented for carrying out catalytic, in particular also exothermic, endothermic or autothermal reactions with optimum yield and selectivity. The system used is a wall-flow monolith which forces a flow from the inlet channel through the porous wall into the outlet channel by reciprocal closure of the gas channels. This is operated such that mass transfer and heat transport are determined virtually exclusively by convection, and diffusion-related thermal conduction phenomena can be neglected.

The present invention relates to a process for carrying out heterogeneously catalyzed gas-phase reactions, in particular including exothermically, autothermically and/or endothermically proceeding reactions, with high selectivity and yield in conjunction with an increase in energy efficiency.

Controlling the process temperature is one of the central problems in carrying out catalytic reactions in a fixed-bed reactor. This holds, in particular, for strongly exothermic or strongly endothermic reactions. In many cases, the desired reaction proceeds only in a narrow temperature window. At temperatures below the temperature window, the reaction rate is low; at temperatures above the temperature window, by contrast, secondary reactions occur and reduce the selectivity. A constant temperature profile in the reactor can be achieved for exothermic and endothermic reactions only when heat is removed from the reactor or supplied to the reactor, or when care is taken to provide good heat transport inside the reactor.

In any form of heat evolution, as low as possible a temperature profile is desired during the reaction, since it is thereby possible to reduce damage to the catalyst by so-called hot spots. Furthermore, the avoidance of temperature differences permits an increase in the turnover-selectivity behaviour, since kinetic parameters can thus be considered as constant, and other process parameters can then be better optimized. In addition, exothermic reactions are always particularly energy efficient whenever the reactor concept permits the heat of reaction evolved to be used for the continuous reaction.

Technically important examples of exothermic reactions which are carried out in a fixed bed reactor and whose yield and selectivity depend decisively on good control of the reaction temperature are partial oxidations, chlorinations and aromatizations of hydrocarbons. Important examples of endothermic reactions are the water gas shift reaction, reforming reactions and dehydrogenations. It is chiefly autothermal reforming reactions which are known as autothermal processes. There is, in addition, the coupling of exothermic and endothermic reactions such as, for example, in synthesis of styrene or of formaldehyde. All forms of catalytic combustion are examples of strongly exothermic reactions with the use of the evolved heat.

Various processes are known for controlling the reaction temperature in a catalytic fixed-bed reactor. A range of processes aims at improving the removal of heat and/or the supply of heat. To this end, the catalyst can, for example, be packed into tubes which are flowed around by a heat exchanger medium. In the case of bulk material reactors, it is also possible for pipe coils with the heat carrier to be placed inside the bed. A further method consists in carrying out the reaction in a series of adiabatic reactors respectively having interposed heat exchangers.

It has also been proposed to carry out the catalytic reactions in monolithic reactors, so-called honeycomb bodies as are used on a large scale in motor vehicle exhaust gas cleaning. With these, the heat exchange can be intensified by circulating a cooling liquid in some channels of the monolith (U.S. Pat. No. 6,143,943). The heat transfer to the outer wall of the reactor can be improved by producing the monolith from a material with good thermal conductivity (US2002/0038062A1). Monolithic carriers for catalytic combustion reactions are known as possible reaction systems from DE2637111 inter alia.

DE 196 53 991 A1 describes a monolithic counterflow reactor for carrying out endothermic catalytic reactions. The counterflow reactor has parallel heating and reaction channels. The reaction channels are coated on their inner walls with a catalyst for the catalytic conversion to be carried out, while the heating channels have on their inner walls a catalyst for the catalytic combustion of a combustion gas/air mixture. This reactor is not suitable for carrying out selective reactions, since the temperature along the reactor rises from 150° C. at the input of the reactor up to 1000° C. in the middle, and then drops to approximately 150° C. again as far as the output.

In addition to the reaction temperature, the dwell time in the reactor also has a decisive influence on yield and selectivity. A series of partial oxidation reactions and dehydrogenations can be carried out in so-called millisecond reactors (J. Krummenacher, L. D. Schmidt, J. Catalysis, 2004, 222, 429-438). In this case the contact time in the reactor is selected to be so short that the desired reactions proceed with high selectivity. The slower total oxidation of the reactants is thereby avoided. The millisecond reactors frequently consist of monoliths which are flowed through by the reaction mixture at so high a velocity that the dwell time in the reactor is of the order of magnitude of a few milliseconds. A limiting factor in the application of millisecond reactors are the diffusion limitations in the channels of the monoliths. The effect of these is that no longer all the reactant molecules come into contact with the surface, and so only a low turnover can be attained. Because of the extensive production of heat on a small reactor volume, the control of the reaction temperature in the case of millisecond reactors constitutes a particular problem. A solution to this problem consists in coupling exothermic and endothermic reactions and having them proceed in adjacent channels of a plate or monolith catalyst (G. A. Deluga, J. R. Salge, X. E. Verykios, L. D. Schmidt, Science, 2004, 303, 993-997).

EP1300193A1 proposes cleaning the exhaust gases of internal combustion engines by leading the exhaust gases through the catalytic coating. By way of example, it is possible to this end to use a so-called wall-flow filter whose channel walls are coated with a catalyst.

Wall flow filters are known from their application in exhaust gas aftertreatment systems, where they are used as soot particle filters. They are mostly monolithic honeycomb bodies which consist of a multiplicity of channels running parallel and provided with a square cross section, their walls being produced at least partially from a porous material which can be flowed through, and their channels being alternately sealed at the inflow and at the outflow ends. The result of this is to force a flow through the partition walls between the channels of the monolith, since gas flowing through can pass into the system only through the inlet channels and escape only through the outlet channels. The channel or cell density of these channels is typically between 10 and 150 cm², while the wall thickness is in the 0.1 to 0.5 mm range.

It is common to modellings of the targeted combustion of the deposited soot particles (regeneration) of such filter systems that, since the work of Bissett et al. (Chemical Engineering Science, 1984, 39, 1233), they are based on considerations of a single-channel system comprising half of the inlet channel, the porous wall and half of the outlet channel as representative of the entire system. 2D modelling of such systems with porous walls that have a catalytic property is known, for example, from Votsmeier et al. (Appl. Cat. B: Environmental, 2007, 70, 233-240).

As described, for example in EP1355048, the actual catalytically active material can be applied in the form of a wash coat to the channels of the wall-flow filter. Additionally or as an alternative, however, introducing the catalyst uniformly into the porous wall is also possible (DE102004040548).

A problem encountered in applying the abovementioned wall-flow filter to catalytically proceeding reactions is that a mass transfer and heat transport from the porous wall onto the reactant stream in the inlet channel can come about. A concentration and temperature gradient can be formed in this way along the channels of the inlet channel. This is counterproductive for the actual conversion, since the strongly differing dwell times and the temperature increase with the channel length can lead to a low selectivity and conversion rate.

It is an object of the present invention to specify a process for carrying out exothermic, endothermic and autothermally heterogeneously catalytic, chemical gas-phase reactions with which such disadvantages like heat transport do not occur. In particular, the process should permit the possibility of carrying out the reaction between gaseous educts with high selectivity, yield and energy efficiency.

This and further objects resulting in an obvious way from the prior art are achieved by a process having the features of the present Claim 1. Preferred refinements of the present process are to be found in the subclaims dependent on Claim 1.

The object set is arrived at extremely surprisingly, but no less advantageously for that, by virtue of the fact that a process for carrying out heterogeneously catalyzed gas-phase reactions for the synthesis of organic molecules is carried out in a wall-flow filter as reactor, in the case of which the catalyst is embedded in the pores of the partition walls of the filter and the channel diameter (d) of the wall-flow filter, the material and pore diameter of the latter and the inflow velocity (u_{rad}) of the reactant gas stream are selected such that a radial Péclet number (Pe_{rad}) of ≧10 results, and furthermore the channel length (1) is selected such that a laminar gas flow prevails under the given conditions inside the channels.

The present invention is directed solely to heterogeneously catalyzed gas-phase reactions for the synthesis of organic molecules. While wall-flow filters are nowadays common as particular filters in exhaust gas cleaning devices, in particular in motor vehicles and power plants, the reactions occurring herein are not seen as producing organic molecules. To the contrary organic molecules are destroyed through such exhaust gas cleaning devices and more or less harmless inorganic compounds like CO₂, CO, NO, NO₂ or H₂O are produced instead. A further differentiating fact between instant processes and exhaust gas cleaning, in particular in motor vehicles, is the fact that the operating conditions differ substantially from those of the particle filter operated in motor vehicle or stationary power plant exhaust gas catalysis, where there is as a rule a maximum space velocity of 100000 h⁻¹ to 120000 h⁻¹, which corresponds to a Péclet number of approximately 5 for the typical dimensions of the systems which come to be applied. In mentioned applications it is desirably to have extremely high space velocities to keep backpressure problems down to a minimum.

(u_{rad}) determines the radial gas velocity and is equal to the velocity of the gas which flows through the wall of the wall-flow filter (u_{wall}). This variable is linked to the velocity of the gas flow entering the filter (vᵢₙₗₑₜ) according to equation 6.

An analysis of the differential equations describing the system (see example section) shows that in the case of a negligible thermal conduction in a radial direction between the inlet channel and wall of the wall-flow filter the dimension less Péclet number (Pe) determines the setting of a temperature gradient along the inlet channel of the wall-flow filter. Operating the reactor with negligible temperature gradients in a radial direction can surprisingly be attained whenever the radial Péclet number Pe_{rad} of ≧10, preferably ≧15, more preferably ≧17, still more preferably ≧19 and most preferably ≧20 prevails.

The Péclet number (according to Jean Claude Eugène Péclet) is a dimensionless characteristic which in thermodynamics reflects the ratio of convectively transported to conducted heat quantity. It corresponds to the product of Reynolds number Re and Prandtl number Pr and is shortened to Pe.

Customary definitions of the Péclet number are:

$$Pe = \frac{v \cdot d}{a} = \frac{v \cdot d \cdot ρ \cdot {C}_{p}}{λ} = Re \cdot Pr \qquad Eq . 1$$

Where:
a—thermal diffusivity (in SI-units: m²/s)
λ—thermal conductivity (in SI-units: W/(mK))
ρ—density (in SI-units: kg/m³)
Cₚ—specific thermal capacity (in SI-units: J/(kgK))
d—characteristic length (in SI-units: m)
v—velocity (in SI-units: m/s)

In the mass transfer, the Péclet number is defined analogously using the Schmidt number Sc:

$$Pe = \frac{v \cdot d}{D} = Re \cdot Sc \qquad Eq . 2$$

Where:
D—diffusion coefficient (in SI-units: m²/s)
d—characteristic length (in SI-units: m)
v—velocity (in SI-units: m/s)

With reference to the wall-flow filter, these equations can be reformulated as:

$$\begin{matrix}{Pe}_{rad} = \frac{{u}_{wall} \cdot {d}_{channel} \cdot {ρ}_{gas} \cdot {Cp}_{gas}}{{λ}_{gas}} \\ or & Eq . 3 \\ {Pe}_{rad} = \frac{{u}_{wall} \cdot {d}_{channel}}{{D}_{gas}} & Eq . 4\end{matrix}$$

D—diffusion coefficient (in SI-units: m²/s)
λ—thermal conductivity (in SI-units: W/(mK))
ρ—density (in SI-units: kg/m³)
Cₚ—specific thermal capacity (in SI-units: J/(kgK))
d—characteristic length (in SI-units: m)
u—radial velocity (in SI-units: m/s).

Equation 4 is applied when the mass transfer coefficient (D_{gas}) becomes large. This is the case, for example, whenever the volume concentrations of the gaseous substances to be reacted are large, or work is carried out in the low pressure region. In the normal case, however, it holds that:

$${D}_{gas} < \frac{{λ}_{gas}}{{ρ}_{gas} \cdot {Cp}_{gas}}$$

In this case, the input of equation 3 predominates, and it is this that is exclusively applied. This holds true, in particularly, for the instant invention where it is deemed that Eq. 4 can be neglected over Eq. 3.

Furthermore, in order to satisfy the conservation law, the incoming volume flow must correspond to the outgoing volume flow.

Vᵢₙ=Vₒᵤₜ Eq. 5
V—volume (in SI-units: m³)

From this, the channel length for the inflow velocity v_{channel} set and the wall velocity u_{wall} in the case of the channel periphery d_{channel} defined by the diameter U_{channel} is yielded as

$${v}_{inlet} \cdot {A}_{inflow surface} = {A}_{outflow surface} \cdot {u}_{wall} ⇒ {l}_{channel} = \frac{{v}_{inlet} \cdot {A}_{inflow surface}}{{U}_{channel} \cdot {u}_{wall}} \qquad Eq . 6$$

v—velocity (in SI-units: m/s)
A—surface (in SI-units: m²)
U—periphery (in SI-units: m²)
u—radial velocity (in SI-units: m/s)
v—radial velocity (in SI-units: m/s).

A further essential condition for dimensioning the wall-flow filter is the fact that the gas stream runs in laminar fashion in the channels of the filter, since the turbulence otherwise occurring leads to undesired back mixtures. It follows that the Reynolds number Re with

$$Re = \frac{{v}_{channel} \cdot {d}_{channel}}{{u}_{gas}} \qquad Eq . 7$$

must be ≦2300.
υ—kinematic viscosity (in SI-units: m²/s)
d—characteristic length (in SI-units: m)
v—velocity (in SI-units: m/s)

The Reynolds number Re is advantageously at ≦1000, preferably at ≦750, with very particular preference at ≦500, and with exceptional preference at ≦250. Furthermore, it is advantageous for the flow into the wall to be as uniform as possible in order to minimize the width of the dwell time distribution in the catalytic wall. Under the conditions in accordance with the claims, it is the case that the mass transfer and heat transport in the wall-flow filter during the reaction proceeds at ≧95% on the basis of convective processes. The convective component is preferably ≧96%, particularly preferably ≧97%, and very particularly preferably ≧98%. This leads to an exceedingly effective energy utilization during the reaction, and permits the catalysis to be operated with optimum yield and selectivity, since the temperature gradient in the radial direction runs very homogeneously in the channel of the wall-flow filter during the reaction, and neither disadvantageous temperature peaks nor temperature troughs occur.

Inflow effects and a possible constriction of the flow in the inlet area can here lead to instances of local turbulence and/or greatly increased flow velocities. Something similar is also to be found at the end of the inlet channel in the vicinity of the closure. With wall-flow filters, a channel length selected to be excessively short can therefore give rise to a non-uniform flow into the catalytically active wall. For chemical conversions on a technical scale, a channel length of the wall-flow filter of 3-32 cm is to be recommended, a length of 4-25 cm is advantageous, and one of 5-20 cm is particularly preferred. Most preferred are wall-flow filter having a channel length of approximately 3, 4, 5, 6, 7, 8, 9, 10 cm.

The material of the wall-flow filter used according to the invention should permit the pressure loss across the filter to be kept as low as possible. This statement relates to the wall-flow filter already provided with catalysts and completely ready to operate. The pressure loss across the wall-flow filter should therefore be less than 50% referred to the input pressure. A pressure loss of less than 35% is advantageous, one of less than 25% is more preferred, and one of less than 15% is very particularly preferred across the filter. In an exceedingly advantageous case, a pressure loss of less than 10% is achieved with reference to the input pressure and in the most favourable case it is a pressure loss of less than 5% which is achieved referred to the input pressure.

As mentioned, the catalyst carrier is designed as a wall-flow filter. According to the invention, the catalytically active material is embedded in the porous partition walls in a finely distributed fashion. Suitable processes for this purpose are familiar to the person skilled in the art (DE102004040548). Thus, as flow takes place through the partition walls there is automatically an intensive contact between the reaction mixture and the catalyst. Owing to the forced flow through the catalyst material, it is also possible for reactions which require a very short dwell time in the reactor to be carried out without restriction by mass transfer phenomena.

In addition, a further catalyst can be applied to the partition walls in the form of a coating, for example as a wash coat. This can serve the purpose of forcing specific preliminary reactions, or allowing a reaction cascade to proceed. However, the precatalyst can also serve to activate the actual reaction partners, which can then react specifically in the activated state in the wall with the second catalyst.

In the case of the reactor geometry described, the catalytic reaction takes place during flow through the partition walls. The surface area of the partition walls coated with catalyst is always a multiple of the entry surface in the case of honeycomb bodies. Correspondingly, the velocity of the reaction gases as they flow through the porous partition walls is also lower by a multiple than the velocity of flow in the inflow and outflow channels. Depending on the nature of the thermal conductivity of the wall, the low velocity of flow in the wall, and the short path length for passing through the wall lead to a very intensive heat exchange between the reaction mixture entering the wall and the converted gases at the exit from the wall. In the case of gaseous reactants, the heat transport onto the incoming reaction mixture is further improved by the thermal conductivity of the wall material, which is high by comparison with the gas. As catalyst carrier, the reactor is therefore preferably produced from a porous, ceramic, wall-flow filter material of high thermal conductivity. Only a slight temperature difference already obtains here in the case of operation in accordance with the invention. A high thermal conductivity then leads to a further compensation thereof. In the most favourable case, the material should therefore have a coefficient of thermal conductivity in W/(m·K) of >0.5, at room temperature, preferably >1, with particular preference of >10, and with very particular preference of >15.

The porosity of the material can be set specifically. In co-operation with the applied pressure, the porosity of the wall-flow filter also influences the dwell time of the reactants and reaction products. The porosity should advantageously have a ratio of hollow body to solid material above 35% m³m⁻³, with particular preference above 50% m³m⁻³, and with very particular preference above 65% m³m⁻³. The carrier material is preferably selected from the group consisting of cellular ceramics. Preference is given to cordierite or silicon carbide as catalyst carriers. The person skilled in the art is very well versed in the production of such wall-flow filters (WO/2007/014562). Preference is particularly given to metal wall-flow filters such as are known, for example, from WO/2000/072944.

As indicated, the person skilled in the art is skilled in the production of the catalytically reactive wall-flow filter (DE102004040548). However, one advantageous embodiment for producing the filter used is that in which the catalytically active substances are firstly deposited on a carrier oxide, and the carrier oxide is deposited in a finely distributed fashion in the pores of the porous wall of the wall-flow filter. Alternatively, it is possible to use wall-flow filters in which the catalytically active substances, if appropriate, are deposited, together with further substances acting as promoter, directly on the porous wall.

The thickness of the catalytically active wall is basically defined firstly from the dwell time required by the reaction kinetics at process temperature as

d_{wall}=u_{wall}·τ Eq. 8

The wall thickness should therefore be minimized, reactions with a dwell time of ≦1 s having proved to be advantageous, reactions with a dwell time of ≦0.1 s having proved to be very advantageous, reactions with a dwell time of ≦0.01 s having proved to be exceedingly favourable and reactions with a dwell time of ≦0.001 s having proved to be extremely advantageous.

The process described is in this case particularly suitable for reactions in which, on the one hand, energy efficiency which is as high as possible is required. It is therefore preferably used for reactions from the group of the catalytic combustions, the methane combustion (http://www.chemie.uni-marburg.de/˜weitzel/lehre/ws2006/kinetik/Methan-Verbrennung.pdf) being regarded as particularly preferred from this group (compare Table 3 in this regard).

Furthermore, the process is suitable for carrying out heterogeneously catalyzed gas phase reactions with high requirements placed on the selectivity of the reaction (compare Table 1 and Table 2 in this regard). Advantageous gas phase reactions for the process illustrated here are ones such as do not exceed an exothermy of 1000 kJ mol⁻¹ referred to the entire gas stream as regards a complete reaction referred proceeding in the preferably adiabatically operated wall-flow filter. Preferred exothermic complete reactions have a negative heat tone of <800 kJ mol⁻¹, with further preference <500 kJ mol⁻¹ and, very particularly, of <200 kJ mol⁻¹. This results from the fact that otherwise the inflowing gas stream must be cooled disproportionately strongly in advance (given 200 kJ mol⁻¹, a temperature difference of above 6000° C. would result), or the gas stream would need to be very strongly diluted.

The inventive process is strongly preferred for those processes in which exothermic and endothermic reactions are coupled. This preferably relates to the synthesis of formaldehyde and strongly preferred autothermal processes of reforming reaction such as, for example, the autothermal reforming reaction of methane.

Further suitable reactions are set forth in the following Tables 1, 2 and 3, respectively.

TABLE 1
Target products of suitable exo- or endothermic secondary reactions for
increasing selectivity in conjunction with energy efficiency

Acetone (ox. Dehydr.)
Acetone (Wacker-Höchst)
Acrolein (Sohio)
Acrylic acid
Aliphatic fluorine compounds
(any form including with other
halogens such as, for example,
chlorine)
Anthraquinone
Benzoic acid
Butanals (n- and iso-)
Cyclohexanol
Acetic acid
Ethylene oxide
Formaldehyde
TPA
Vinyl chloride

TABLE 2
Suitable autothermally conducted reactions for increasing conversion
and/or selectivity

Autothermal reforming
reaction of each hydrocarbon
Hydrocyanic acid production
(Andrussow)
Formaldehyde (autothermal)
cat. Ox. of NH₃ to NO
(Ostwald process)
Methanation
Styrene process

TABLE 3
Suitable exothermic reactions for increasing energy efficiency

Catalytic combustion of each
hydrocarbon

In the case of high space velocities/short dwell times of reactants over the catalytically active substance, the heat transport and mass transfer are determined practically exclusively by convection. Only prominent concentration gradients form along the interface, but no radial concentration profiles form inside the inlet channel. Under the inventive conditions, it is possible to implement in monolithic wall-flow filter systems
shorter dwell times in conjunction with full conversion, and
to define a very narrow dwell time window, which leads to increases in selectivity, in particular for secondary reactions.

By expanding the geometry, and thus enlarging the wall diameter, this obtains for any desired systems and independent of absolute dwell times.

By applying the present process, the results achieved can be combined with equivalent advantages in the evolution of heat and heat transport. Firstly, the uniform distribution of the reaction process leads to a uniform evolution of heat or to a heat loss. This is favourable precisely for autothermal reactions. Secondly, the heat in the zone of the actual reaction process remains owing to minimization of the conduction of heat in the inlet channel, and can thus be used for continuous operation. This is achieved by virtue of the fact that the inflow to the wall over the greater part of the system takes place with a radial Péclet number ≧10 with regard both to mass transfer and to heat transport.

For one thing, this leads to circumvention of limitations with regard to the macroscopic mass transfer, and at the same time ensures a very exact dwell time setting. Furthermore, a uniform conversion yields a homogeneous local evolution of heat (or removal of heat) which, in turn, leads to a very homogeneous temperature profile over the entire wall owing to the very slight conduction of heat back into the inlet channel.

Together, the two effects result, through the exact setting of temperature and dwell time in the entire catalytically active zone, in a rise in yield of the desired products in conjunction with reactions which are more selective. Furthermore, the very uniform production (or reduction) of heat leads to an increased energy efficiency of the system. The yields of autothermally conducted processes can also be increased thereby.

When summarized in a diagram, the variables are represented in dependence on one another such that all conditions are fulfilled within the zone enclosed by them (FIG. 7—design diagram for the exemplary values Pe=20 and Re=500).

The following example and the figures serve the purpose of further explanation of the invention. In the drawing:

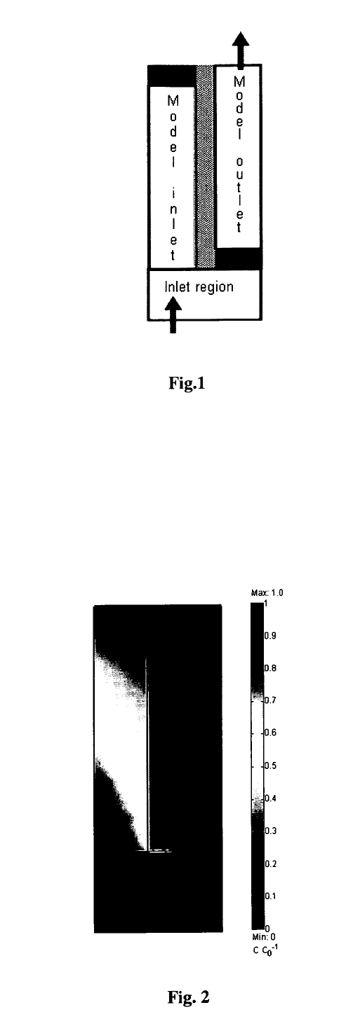

FIG. 1: shows the geometrical model used as a basis for the simulations,

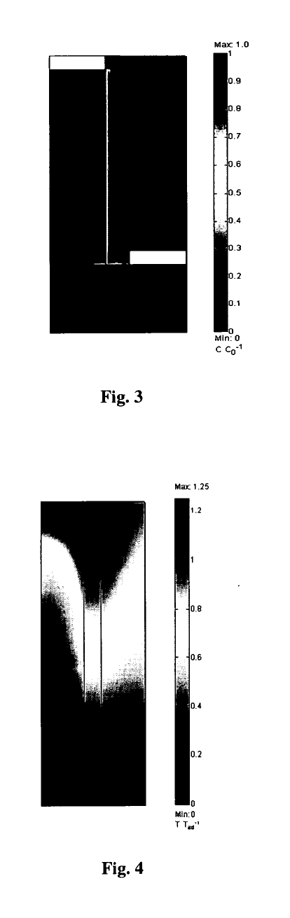

FIG. 2: shows a calculated concentration profile for an educt for non-inventively set wall-flow filters,

FIG. 3: shows a calculated concentration profile for an educt for inventively set wall-flow filters,

FIG. 4: shows a calculated temperature profile for non-inventively set wall-flow filters,

FIG. 5: shows a calculated temperature profile for inventively set wall-flow filters,

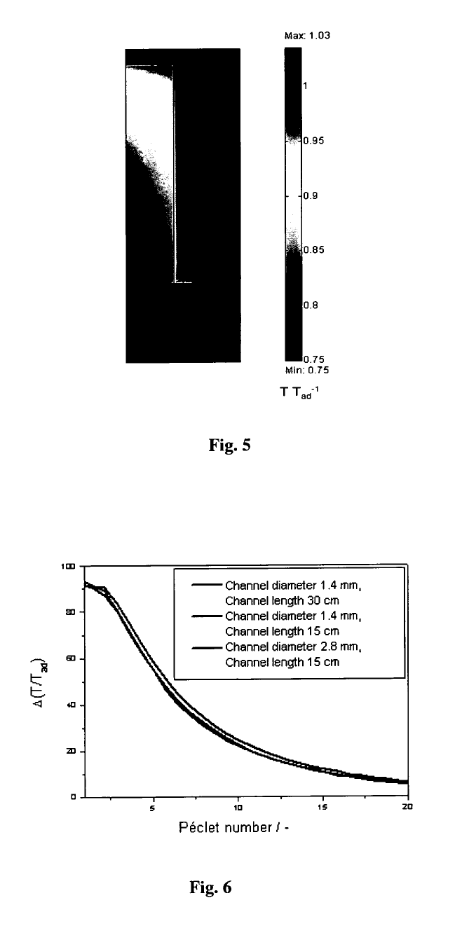

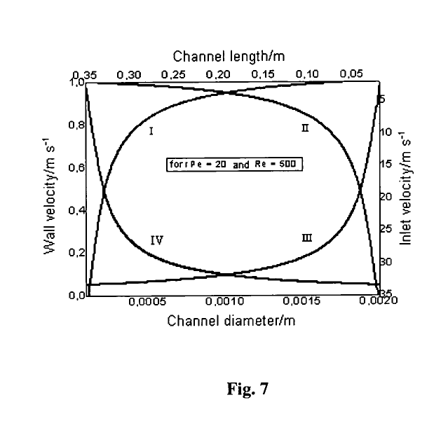

FIG. 6: shows a relative temperature gradient inside the catalytically active wall referred to the adiabatic temperature increase as a function of the Péclet number for various geometries, and

FIG. 7: shows a design diagram for the exemplary values Pe=20 and Re=500 with the wall velocity as a function of channel diameter (IV), with the channel length as a function of wall velocity (III), with the channel length as a function of inlet velocity (II), and with the channel length as a function of wall velocity (I).

### EXAMPLES

### Modelling Fundamentals

Given the introduction of suitable boundary conditions for utilizing the symmetrical properties as illustrated in FIG. 1, two-dimensional models of a so-called wall-flow filter comprise half the inlet channel, the porous wall and half the outlet channel. In order to depict inflow effects, an inlet region is additionally placed in front of the actual system.

FIG. 1: Graphical representation of the 2-D model.

A laminar flow can be assumed in the inlet region, inlet and outlet channels on the basis of the calculated local Reynolds numbers. It therefore follows as a function of temperature from the local solution of the Navier-Stokes equation (Eq. 9) that:

$$ρ \cdot \frac{\partial v}{\partial t} - ∇ ( ∇ v + {( ∇ v )}^{T} ) + ρ ( v \cdot ∇ ) v + ∇ p = F \qquad Eq . 9$$

The flow through the porous wall is described with the Brinkman equation (Eq. 10), which is an extension of the Darcy Law from which, by an additive term, the shear stress becomes:

$$ρ \cdot \frac{\partial v}{\partial t} - ∇ \cdot η ( ∇ v + {( ∇ v )}^{T} ) - \frac{η}{k} v + ∇ p = F \qquad Eq . 10$$

The respective temperature profile results from the solution of the energy balance (Eq. 11), in which exothermy or endothermy of the reaction can also be embedded by an appropriate expression for the heat source/sink Q:

$$\underbrace{ρ \cdot {c}_{p} \cdot \frac{\partial T}{\partial t}}_{ \begin{matrix}time rate of \\ charge of energy\end{matrix}} = \underbrace{Q}_{ \begin{matrix}energy \\ source therm\end{matrix}} - ∇ \cdot ( \underbrace{- k \cdot ∇ T}_{ \begin{matrix}energy transfer \\ by conduction\end{matrix}} + \underbrace{ρ \cdot {c}_{p} \overline{u} \cdot T}_{ \begin{matrix}energy transfer \\ by convection\end{matrix}} ) \qquad Eq . 11$$

The mass transfer results from the mass balance (Eq. 12), which can include different expressions for the reaction term.

$$\underbrace{\frac{\partial {c}_{i}}{\partial t}}_{ \begin{matrix}time rate of \\ change of concentration\end{matrix}} = \underbrace{{R}_{i}}_{ \begin{matrix}reaction \\ therm\end{matrix}} - ∇ \cdot ( \underbrace{- {D}_{i} \cdot ∇ {c}_{i}}_{ \begin{matrix}mass transfer \\ by diffusion\end{matrix}} + \underbrace{\vec{u} \cdot {c}_{i}}_{ \begin{matrix}mass transfer \\ by convection\end{matrix}} ) \qquad Eq . 12$$

With the exception of wall thickness and wall permeability, the parameters used are regarded as dependent on temperature and conform with Zhang, et al.; the values at the reference temperature of 200° C. are reproduced in Table 4 (F. Zhang, R. E. Flayes, S. T. Kolaczowski, Chem. Eng. Res. Des. 82, 481-489, 2004).

TABLE 4
Values of the parameters used at 200° C.

| | Gas | Wall |
|---|---|---|
| Density | 1.23 kg m³ | 1600 kg m³ |
| Heat capacity | 1026 W s kg⁻¹ K⁻¹ | 952 W s kg⁻¹ K⁻¹ |
| Thermal | 0.032 W s m⁻¹ K⁻¹ | 20 W s m⁻¹ K⁻¹ |
| conductivity | | |
| Diffusion coefficient | 4.3 × 10⁻⁵ m² s1 | 1 × 10⁻⁶ m² s⁻¹ |
| Permeability | | 5.5e−13 m² |

All models used were solved numerically using a finite element process (FEM), use being made for this purpose of the commercially available software FEMLAB 3.1 (COMSOL AB).

FIG. 2 shows the simulated concentration profile of the educt of a non-inventively set system (Pe=2). The reaction takes place in this case at the catalyst, which is located inside the partition wall between inlet and outlet channels. The concentration gradient produced between the inflowing gas stream and the gas in the wall leads to a pronounced back diffusion against the inflow direction of the radial flow. The dwell time in the catalytically active zone is therefore not precisely defined, and this leads in the case of selective reactions to a reduction in the yield of the desired product.

FIG. 3 shows the simulated concentration profile of the educt of an inventively set system (Pe=20, Re=1700). Here, as well, the reaction takes place on the catalyst, which is located inside the partition wall between inlet and outlet channels. In an inventive selection of the process parameters, no back diffusion results in the inlet channel, while equally the inflow to the wall takes place very uniformly over the entire length. This leads to the possibility of a very exact dwell time definition at the catalyst. Moreover, the mass transfer, determined exclusively by convection, has the effect that the macroscopic mass transfer is ensured even given the high space velocity and high pressures.

Furthermore, it emerges with the aid of simulation calculations that given an appropriate design it is possible to use the specified process to carry out an exothermic catalytic reaction with a temperature gradient of less than 10% of the adiabatic temperature increase in conjunction with a conversion of 99%.

FIG. 4 shows the temperature change for a non-inventively set system (Pe=2) along the channels, referred to the adiabatic temperature increase (plotted (T−Tᵢₙₗₑₜ)/ΔT_{adiabatic}) in conjunction with a virtually complete (99.5%) conversion which is uniform over the entire length, T being the temperature, Tᵢₙₗₑₜ the inlet temperature, and ΔT the adiabatic temperature increase. Clearly evident here is the temperature gradient along the wall, which extends over a range of just under 90% relative temperature increase.

FIG. 5 shows the temperature change for a non-inventively set system (Pe=20, Re=1700) along the channels, referred to the adiabatic temperature increase (plotted (T−Tᵢₙₗₑₜ)/ΔT_{adiabatic}) in conjunction with a virtually complete (99.5%) conversion which is uniform over the entire length, T being the temperature, Tᵢₙₗₑₜ the inlet temperature, and ΔT the adiabatic temperature increase. The temperature gradient along the wall now comes out as in the range of 5%, and so the wall temperature is very uniform overall.

FIG. 6 shows once again the temperature gradient produced, as a function of the Péclet number. In this case, the deviation of the locally resulting temperature increase was re-determined, and the difference between the integral wall temperature at the start and at the end of the system was considered (spacing from the respective outer boundary 0.5% of the total channel length). This procedure was executed for three different geometries in order once again to show the independence of the criterion used from dimension.

1. Process for carrying out heterogeneously catalyzed gas-phase reactions for the synthesis of organic molecules in a wall-flow filter as reactor, the catalyst being embedded in the pores of the partition walls of the filter,
wherein,
the channel diameter (d) of the wall-flow filter, the material and pore diameter thereof, and the inflow velocity (u_{rad}) of the reactant gas stream are selected such that a radial Péclet number (Pe_{rad}) of ≧10 results, and furthermore the channel length (1) is selected such that a laminar gas flow prevails inside the channels under the given conditions (Re≦2300).

2. Process according to claim 1,
wherein,
the pressure loss across the wall-flow filter is less than 25% referred to the input pressure.

3. Process according to claim 1,
wherein,
a further catalyst is additionally applied to the partition walls in the form of a coating.

4. Process according to claim 1,
wherein,
the wall-flow filter consists of material with a thermal conductivity
of ≧0.5 W m⁻¹K⁻¹ at room temperature.

5. Process according to claim 1,
wherein,
the reaction is autothermal, exothermic or endothermic.

6. Process according to claim 5,
wherein,
the complete reaction proceeding in the wall-flow filter does not exceed an exothermy of 1000 kJ mol⁻¹ referred to the total gas flow.

7. Process according to claim 2,
wherein,
a further catalyst is additionally applied to the partition walls in the form of a coating.

8. Process according to claim 7,
wherein,
the wall-flow filter consists of material with a thermal conductivity
of ≧0.5 Wm⁻¹K⁻¹ at room temperature.

9. Process according to claim 2,
wherein,
the wall-flow filter consists of material with a thermal conductivity
of ≧0.5 Wm⁻¹K⁻¹ at room temperature.

10. Process according to claim 3,
wherein,
the wall-flow filter consists of material with a thermal conductivity
of ≧0.5 Wm⁻¹K⁻¹ at room temperature.
